(19) United States
(12) Patent Application Publication
(10) Pub. No.: US 2026/0337219 A1
(43) Pub. Date: Oct. 29, 2026

(54) AMORPHOUS COMPOUNDS OF REDUCED BETA-NICOTINAMIDE MONONUCLEOTIDE DISODIUM SALT AMORPHOUS COMPOUND AND THEIR PREPARATION METHODS

(71) Applicant: BONTAC BIO-ENGINEERING(SHENZHEN) CO.,LTD, Guangdong (CN)

(72) Inventors: Tao GUO, Guangdong (CN); Zhigang ZHOU, Guangdong (CN); Qi ZHANG, Guangdong (CN)

(21) Appl. No.: 19/394,593

(22) Filed: Nov. 19, 2025

(30) Foreign Application Priority Data
Nov. 19, 2024 (CN) 2024116507293

(51) Int. Cl. C07H 19/048; C07H 1/00

(52) U.S. Cl. CPC C07H 19/048; C07H 1/00

(57) ABSTRACT

The present disclosure relates to an amorphous compound of a reduced β-nicotinamide mononucleotide disodium salt and a preparation method therefor, and belongs to the field of raw materials for health care products, cosmetics, and food additives. The preparation method of the present disclosure mainly includes five main steps of adding a poor solvent to a concentrated aqueous solution of a reduced β-nicotinamide mononucleotide disodium salt, adding a seed crystal and stirring to generate a precipitate, continuously adding dropwise the poor solvent to further precipitate out the precipitate, filtering, and performing vacuum drying. The prepared reduced β-nicotinamide mononucleotide disodium salt is an amorphous compound, and the obtained amorphous compound does not contain crystal water, has a water content of lower than 5%, and can be stably stored for more than 3 months under air-isolated conditions at a temperature of 20-30 °C, thus having an outstanding stability advantage.

### TECHNICAL FIELD

The present disclosure belongs to the field of raw materials for health care products, cosmetics, and food additives, and particularly relates to an amorphous compound of a reduced β-nicotinamide mononucleotide disodium salt and a preparation method therefor.

### BACKGROUND

Nicotinamide adenine dinucleotide (NAD+) is an essential coenzyme required for more than 500 enzymatic reactions. An increasing number of studies show that increasing an equivalent of NAD+ can obviously improve functions of multiple organs, including liver functions, kidney functions, cardiac functions, and skeletal muscle functions. NAD+ can be synthesized using tryptophan in a de novo biosynthesis pathway, using nicotinic acid (NA) in a preiss-handler pathway, as well as using nicotinamide (NAM), nicotinamide riboside (NR), and nicotinamide mononucleotide (NMN) in a salvage pathway. Particularly, as key NAD+ intermediates, NAM, NR, and NMN have been widely studied due to their potential therapeutic roles in various mouse disease models. Although increasing the level of NAD+ by supplementing intermediates is beneficial to health and extends a lifespan, their pharmacokinetics and metabolic fate in different organs under various disease conditions are still under research. Furthermore, it remains unclear whether supplementing NAD+ precursors can prevent liver injury or hepatic fibrosis by maintaining redox homeostasis. The key NAD intermediates (commonly referred to as “NAD supplements”), such as NMN, have attracted the attention of researchers, investors, and consumers for their significant effects related to disease and aging. Conventional NAD supplements, such as NMN, are oxidized forms of NAD precursors. Studies show that a novel reduced form of an NAD precursor, namely, reduced β-nicotinamide mononucleotide (NMNH), has a better NAD promotion effect than NMN and has other biological functions, such as increasing a cellular antioxidant capacity, reducing fat accumulation, decreasing inflammatory responses, and inhibiting tumor cell growth.

A synthesis process of a reduced β-nicotinamide mononucleotide disodium salt (NMNH) is currently at a laboratory research and development stage and cannot yet achieve industrial production. Main technical difficulties are that first, NMNH is a reduced form of NMN and is easily oxidized by air; and second, most existing preparation methods require the method of vacuum freeze-drying to obtain amorphous solids, resulting in high production costs and an inability to achieve industrial production. NMNH products prepared by existing technologies have problems, such as low purity, poor flowability, easy moisture absorption leading to oily substances, and rapid degradation, accompanied by a certain degree of degradation that usually occurs during freeze-drying, resulting in a low product purity and an inability to meet product application requirements. Due to the poor flowability and the easy moisture absorption, the products cannot be encapsulated, tableted, and shape-formed during preparation processing. A freeze-dried powder obtained by the freeze-drying is needle-like and has a large specific surface area and an increased contact area with air and moisture, leading to an increased degradation rate. Therefore, the development of novel NMNH having good stability and good flowability and being suitable for industrialization has a positive significance.

### SUMMARY

In view of the problems proposed in the background art, a first objective of the present disclosure is to provide a preparation method for a reduced β-nicotinamide mononucleotide disodium salt. The preparation method has the characteristics of simple operation, a high product purity, a high yield, no requirement for the use of high-investment and low-output equipment for freeze-drying, and more easiness in achieving industrialization. A second objective of the present disclosure is to provide a reduced β-nicotinamide mononucleotide disodium salt obtained by the preparation method of the present disclosure. The salt is an amorphous compound, which has two characteristic glass transition temperatures in temperature ranges of 63.69 °C-74.67 °C and 85.52 °C-131.99 °C, with peak values of the two glass transition temperatures being 68.78 °C and 112.93 °C, respectively; has a characteristic melting point temperature at 217.21 °C-241.88 °C, with a peak value of the characteristic melting point temperature being 231.70 °C; and as shown by Raman spectroscopy, has stronger characteristic peaks at Raman shifts (cm⁻¹) of 814 cm⁻¹ and 845 cm⁻¹. The amorphous compound of the reduced β-nicotinamide mononucleotide disodium salt has the advantages of a high purity, a moisture content of less than 5%, a high effective ingredient content, and low hygroscopicity, and has good formability. Compared with a crystalline reduced β-nicotinamide mononucleotide disodium salt, the salt has a lower moisture content and can be stably stored for more than 3 months at a temperature of 20 °C-30 °C, thus having an outstanding stability advantage.

In order to achieve the above-mentioned objectives, the present disclosure is achieved through the following technical solutions.

The preparation method for a reduced β-nicotinamide mononucleotide disodium salt provided by the present disclosure includes the following steps:
(1) using β-nicotinamide mononucleotide as a raw material, thiourea dioxide as a reducing agent, and pure water as a reaction solvent to carry out a reaction under alkaline conditions to obtain an aqueous solution of reduced β-nicotinamide mononucleotide;
(2) separating and purifying the aqueous solution of the reduced β-nicotinamide mononucleotide using a preparative high performance liquid chromatograph;
(3) removing an ammonium salt using a strongly acidic cation exchange resin, performing nanofiltration and concentration with a nanofiltration membrane, and adjusting a pH value with a sodium hydroxide solution to obtain a concentrated aqueous solution of a reduced β-nicotinamide mononucleotide disodium salt;
(4) adding a poor solvent to the concentrated aqueous solution of the reduced β-nicotinamide mononucleotide disodium salt;
(5) stirring and adding a seed crystal;
(6) continuously stirring to generate a suspended precipitate;
(7) continuously adding the poor solvent to increase a yield; and
(8) filtering and drying to obtain an amorphous compound of the reduced β-nicotinamide mononucleotide disodium salt.

Further, in the step (1), an aqueous solution of the β-nicotinamide mononucleotide is first formulated, the thiourea dioxide is added to the solution to initiate the reaction after the pH value is adjusted to 9.5-10.0 by adding an alkali, and the pH value is maintained at 9.5-10.0 during the reaction.

Further, the poor solvent in the step (2) is ethanol; and in the step (4), a temperature of the concentrated aqueous solution of the reduced β-nicotinamide mononucleotide disodium salt is 0 °C-25 °C, and the poor solvent is anhydrous ethanol.

Further, the seed crystal is ribose, glucose, or a reduced β-nicotinamide mononucleotide disodium salt prepared and obtained by using ribose or glucose as a precipitation primer.

Further, a mass percentage content of the reduced β-nicotinamide mononucleotide disodium salt in the concentrated aqueous solution of the reduced β-nicotinamide mononucleotide disodium salt in the step (4) is 5%-15%.

Further, a reaction temperature in the step (1) is 39 °C-44 °C; and a drying temperature in the step (8) is 20 °C-35 °C.

Further, an amount of the poor solvent added in the step (4) is 1.5-4.0 times that of the concentrated aqueous solution of the reduced β-nicotinamide mononucleotide disodium salt; and an amount of the poor solvent added dropwise in the step (7) is 3.5-6.0 times that of the concentrated aqueous solution of the reduced β-nicotinamide mononucleotide disodium salt.

The present disclosure further provides a reduced β-nicotinamide mononucleotide disodium salt obtained by the above-mentioned preparation method.

Further, the reduced β-nicotinamide mononucleotide disodium salt is an amorphous compound, which, as detected by a differential scanning calorimeter after being heated from 10 °C to 300 °C at a heating rate of 10 °C/min, has two characteristic glass transition temperatures at 63.69 °C-74.67 °C and 85.52 °C-131.99 °C, with peak values of the two characteristic glass transition temperatures being 68.78 °C and 112.93 °C, respectively; has a characteristic melting point temperature at 217.21 °C-241.88 °C, with a peak value of the characteristic melting point temperature being 231.70 °C; and as scanned by a laser confocal Raman spectrometer at an excitation wavelength of 532 nm, a grating of 600 lines, an objective lens of 50×, an integration time of 5 seconds, and an accumulation number of times of 3 times, has characteristic peaks at Raman shifts of 814 cm⁻¹ and 845 cm⁻¹ as shown by detection results.

The amorphous compound of the reduced β-nicotinamide mononucleotide disodium salt obtained by the preparation method of the present disclosure is used as a raw material for preparing health care products, cosmetics, and food additives.

Beneficial effects of the present disclosure are as follows.

The reduced β-nicotinamide mononucleotide disodium salt can be prepared by the present disclosure without the need of using freeze-drying equipment. The preparation method is simple in operation, low in cost, and more suitable for industrial production.

The reduced β-nicotinamide mononucleotide disodium salt prepared by the present disclosure is an amorphous compound, which, (1) as detected by a differential scanning calorimeter after being heated from 10 °C to 300 °C at a heating rate of 10 °C/min, has two characteristic glass transition temperatures at 63.69 °C-74.67 °C and 85.52 °C-131.99 °C, with peak values of the two characteristic glass transition temperatures being 68.78 °C and 112.93 °C, respectively; and has a characteristic melting point temperature at 217.21 °C-241.88 °C, with a peak value of the characteristic melting point temperature being 231.70 °C; (2) as scanned by a laser confocal Raman spectrometer at an excitation wavelength of 532 nm, a grating of 600 lines, an objective lens of 50×, an integration time of 5 seconds, and an accumulation number of times of 3 times, has characteristic peaks at Raman shifts of 814 cm⁻¹ and 845 cm⁻¹ as shown by detection results; and (3) has a good appearance, color, and luster, a high purity, a moisture content of less than 5%, a high effective ingredient content, low hygroscopicity, and good formability, and can be stably stored for more than 3 months at a temperature of 20 °C-30 °C.

### BRIEF DESCRIPTION OF THE DRAWINGS

FIG. 1 is an X-ray powder diffraction (XRPD) pattern of an amorphous compound of NMNH of Example 5 of the present disclosure;

FIG. 2 is a nuclear magnetic resonance hydrogen spectrogram of the amorphous compound of NMNH of Example 5 of the present disclosure;

FIG. 3 is a negative-mode mass spectrogram of the amorphous compound of NMNH of Example 5 of the present disclosure;

FIG. 4 is a positive-mode mass spectrogram of the amorphous compound of NMNH of Example 5 of the present disclosure;

FIG. 5 is a thermogravimetric analysis (TGA) spectrogram of the amorphous compound of NMNH of Example 5 of the present disclosure;

FIG. 6 is a TGA spectrogram of a freeze-dried powder of a reduced β-nicotinamide mononucleotide disodium salt of Example 2 of the present disclosure;

FIG. 7 is a differential scanning calorimetry (DSC) spectrogram of the amorphous compound of NMNH of Example 5 of the present disclosure;

FIG. 8 is a DSC spectrogram of the freeze-dried powder of the reduced β-nicotinamide mononucleotide disodium salt of Example 2 of the present disclosure;

FIG. 9 is a Raman spectrogram of the amorphous compound of NMNH of Example 5 of the present disclosure;

FIG. 10 is a Raman spectrogram of the freeze-dried powder of the reduced β-nicotinamide mononucleotide disodium salt of Example 2 of the present disclosure;

FIG. 11 is an XRPD pattern of the freeze-dried powder of the reduced β-nicotinamide mononucleotide disodium salt of Example 2 of the present disclosure;

FIG. 12 is a high performance liquid chromatography (HPLC) spectrogram of the amorphous compound of NMNH of Example 5 of the present disclosure.

### DETAILED DESCRIPTION OF THE EMBODIMENTS

In order to make the objectives, technical solutions, and beneficial effects of the present disclosure clearer, the technical solutions of the present disclosure are described in detail below. Obviously, examples described are only a part of the examples of the present disclosure, rather than all of the examples. Based on the examples of the present disclosure, all other examples obtained by those skilled in the art without making creative labor shall fall within the scope of protection of the present disclosure.

A preparation method for a reduced β-nicotinamide mononucleotide disodium salt includes the following steps:
(1) first formulating an aqueous solution of β-nicotinamide mononucleotide, adding thiourea dioxide to the solution to initiate a reaction after adjusting a pH value to 9.5-10.0 by adding an alkali, and maintaining the pH value of the reaction solution at 9.5-10.0 during the reaction to obtain an aqueous solution of reduced β-nicotinamide mononucleotide;
(2) separating and purifying the aqueous solution of the reduced β-nicotinamide mononucleotide using a preparative high performance liquid chromatograph;
(3) removing an ammonium salt using a strongly acidic cation exchange resin;
(4) performing nanofiltration and concentration with a nanofiltration membrane, and adjusting the pH value with a sodium hydroxide solution to obtain a concentrated aqueous solution of a reduced β-nicotinamide mononucleotide disodium salt;
(5) controlling the concentrated aqueous solution of the reduced β-nicotinamide mononucleotide disodium salt at a temperature of 0 °C-25 °C and a mass percentage concentration of 5%-15%, and adding anhydrous ethanol, with a mass of the ethanol added being 1.5-4.0 times that of the concentrated aqueous solution of the reduced β-nicotinamide mononucleotide disodium salt;
(6) stirring and adding a seed crystal, with the seed crystal being ribose, glucose, or a reduced β-nicotinamide mononucleotide disodium salt prepared and obtained by using ribose or glucose as a precipitation primer;
(7) continuously stirring to generate a suspended precipitate;
(8) continuously adding a poor solvent to increase a yield, with a mass of the poor solvent added dropwise being 3.5-6.0 times that of the concentrated aqueous solution of the reduced β-nicotinamide mononucleotide disodium salt; and
(9) filtering and drying at 20 °C-35 °C to obtain an amorphous compound of the reduced β-nicotinamide mononucleotide disodium salt.

In order to describe the present disclosure more clearly, the following detailed description is provided through the following examples.

### Example 1 Preparation of a Freeze-Dried Powder of a Reduced β-Nicotinamide Mononucleotide Disodium Salt (with the Above Step (3) Omitted and a Product Obtained by a Freeze-Drying Method)

Preparation of an aqueous solution of a reduced β-nicotinamide mononucleotide disodium salt: 15 L of pure water was added into a 50 L glass reactor and heated to 40 °C, 1.52 kg of NMN was added into the glass reactor, and under stirring conditions, a pH was adjusted to 9.5-10.0 with a 10 mol/L sodium hydroxide solution until the resulting solution was dissolved and clarified. 0.80 kg of thiourea dioxide was added into the glass reactor to carry out a reaction at a temperature controlled at 39 °C-44 °C under stirring for 5 hours. During the reaction, the pH was adjusted with a 10 mol/L sodium hydroxide solution, wherein the pH was controlled at 9.0-10.0. After completion of the reaction, the pH was adjusted to 10.5-11.0 with a 10 mol/L sodium hydroxide solution. A preparative high performance liquid chromatograph (a diameter of a chromatographic column was 15 cm, and a column volume was 6 L; a packing material was a polystyrene reversed-phase packing material, with a packing material model of Unis P40-300, provided by NanoMicro Technology Co., Ltd.; mobile phases were water and ethanol; a loading volume of a reaction solution each time was 1.5 L-2.0 L, and a product was eluted with pure water after loading; fractions with a purity of greater than 98.50% were collected in sections to serve as qualified products and sent to the next process step, and fractions with a purity of less than 98.50% were collected to serve as unqualified products and sent to the reaction solution for re-purification, with a time consumption of 25-30 minutes for each purification; and after completion of the purification for 10 times, the reaction solution was eluted with a 20% ethanol aqueous solution to remove impurities on the chromatographic column, the chromatographic column was rinsed with pure water, and then the reaction solution was continuously loaded for purification, with a wavelength of an ultraviolet detector being 340 nm) was used for purification and separation to obtain an aqueous solution of reduced β-nicotinamide mononucleotide. Nanofiltration and concentration were performed with a nanofiltration membrane to 14 L, and during the nanofiltration and the concentration, the pH value was adjusted with a 10 mol/L sodium hydroxide solution and maintained at 10.5-11.0 to obtain a concentrated aqueous solution of a reduced β-nicotinamide mononucleotide disodium salt, which contained 1.66 kg of the reduced β-nicotinamide mononucleotide disodium salt as detected by a high performance liquid chromatograph (with a C18 chromatographic column, a detection wavelength of 340 nm, an external standard method for detection, and a standard product purchased from Hubei WeiShi Chemical Reagent Co., Ltd.).

Preparation of a freeze-dried powder: The concentrated aqueous solution of the reduced β-nicotinamide mononucleotide disodium salt was fed into a 2 m² vacuum freeze dryer for freeze-drying, then pre-frozen at −50 °C for 2 hours, and gradually heated to 30 °C for 72 hours to obtain 1.69 kg of an amorphous pine needle-like light yellow powder, that is, an amorphous freeze-dried powder of the reduced β-nicotinamide mononucleotide disodium salt, being fluffy like cotton fluff. The freeze-dried powder of the reduced β-nicotinamide mononucleotide disodium salt remaining on a drying tray becomes viscous after exposure to the air for half an hour and becomes an oily substance after one hour. A weight loss on drying of the freeze-dried powder of the reduced β-nicotinamide mononucleotide disodium salt at 120 °C is 6.48%, a sodium ion content detected by an ion chromatograph is 11.05%, and the sodium ion content is calculated to be 11.81% after deducting moisture. The obtained freeze-dried powder has a pungent ammonia odor. Detection with the ion chromatograph shows that no free ammonium ion residue is detected, inferring that ammonium ions replace a part of sodium ions to form a salt with NMNH.

### Example 2 Preparation of a Freeze-Dried Powder of a Reduced β-Nicotinamide Mononucleotide Disodium Salt (with a Product Obtained by a Freeze-Drying Method)

Preparation of an aqueous solution of a reduced β-nicotinamide mononucleotide disodium salt: 15 L of pure water was added into a 50 L glass reactor and heated to 44 °C, 1.55 kg of NMN was added into the glass reactor, and under stirring conditions, a pH was adjusted to 9.5-10.0 with a 10 mol/L sodium hydroxide solution until the resulting solution was dissolved and clarified. 0.82 kg of thiourea dioxide was added into the glass reactor to carry out a reaction at a temperature controlled at 39 °C-44 °C under stirring for 6 hours. During the reaction, the pH was adjusted with a 10 mol/L sodium hydroxide solution, wherein the pH was controlled at 9.0-10.0. After completion of the reaction, the pH was adjusted to 10.5-11.0 with a 10 mol/L sodium hydroxide solution. A preparative high performance liquid chromatograph (a diameter of a chromatographic column was 15 cm, and a column volume was 6 L; a packing material was a polystyrene reversed-phase packing material, with a packing material model of Unis P40-300, provided by NanoMicro Technology Co., Ltd.; mobile phases were water and ethanol; a loading volume of a reaction solution each time was 1.5 L-2.0 L, and a product was eluted with pure water after loading; fractions with a purity of greater than 98.50% were collected in sections to serve as qualified products and sent to the next process step, and fractions with a purity of less than 98.50% were collected to serve as unqualified products and sent to the reaction solution for re-purification, with a time consumption of 25-30 minutes for each purification; and after completion of the purification for 10 times, the reaction solution was eluted with a 20% ethanol aqueous solution to remove impurities on the chromatographic column, the chromatographic column was rinsed with pure water, and then the reaction solution was continuously loaded for purification, with a wavelength of an ultraviolet detector being 340 nm) was used for purification and separation to obtain an aqueous solution of reduced β-nicotinamide mononucleotide. The obtained aqueous solution of the reduced β-nicotinamide mononucleotide was subjected to ammonia removal and purification operation using a 001X7 strongly acidic cation exchange resin (a resin volume was 6 L, the solution was treated with a 0.5-1 mol/L sodium hydroxide solution before the purification to obtain a sodium form and then rinsed with purified water to a pH of 7-9 to load the aqueous solution of the reduced β-nicotinamide mononucleotide, and the loaded solution was eluted with pure water at a flow rate of 1-1.5 times column volume/hour). After the ammonia removal and the purification using the 001X7 strongly acidic cation exchange resin, a purified solution of the reduced β-nicotinamide mononucleotide was obtained. Nanofiltration and concentration were performed with a nanofiltration membrane to 16 L, and during the nanofiltration and the concentration, the pH value was adjusted with a 10 mol/L sodium hydroxide solution and maintained at 10.5-11.0 to obtain a concentrated aqueous solution of a reduced β-nicotinamide mononucleotide disodium salt, which contained 1.68 kg of the reduced β-nicotinamide mononucleotide disodium salt as detected by a high performance liquid chromatograph (with a C18 chromatographic column, a detection wavelength of 340 nm, an external standard method for detection, and a standard product purchased from Hubei WeiShi Chemical Reagent Co., Ltd.).

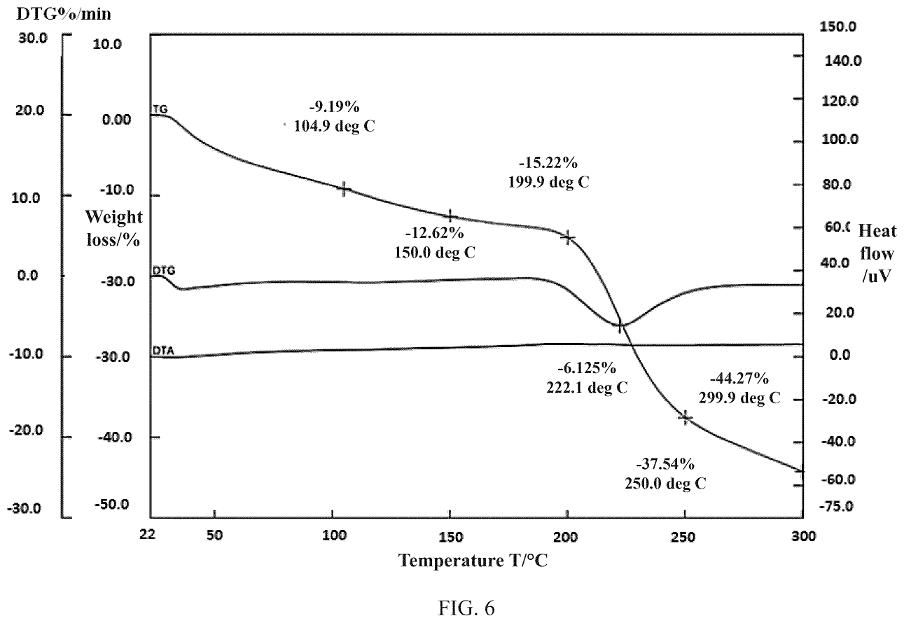

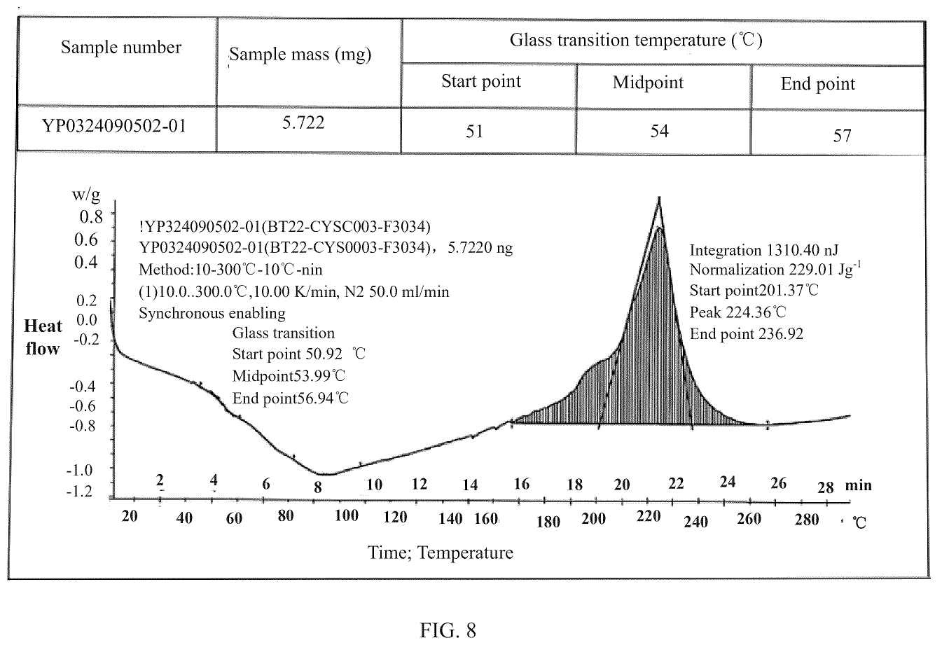

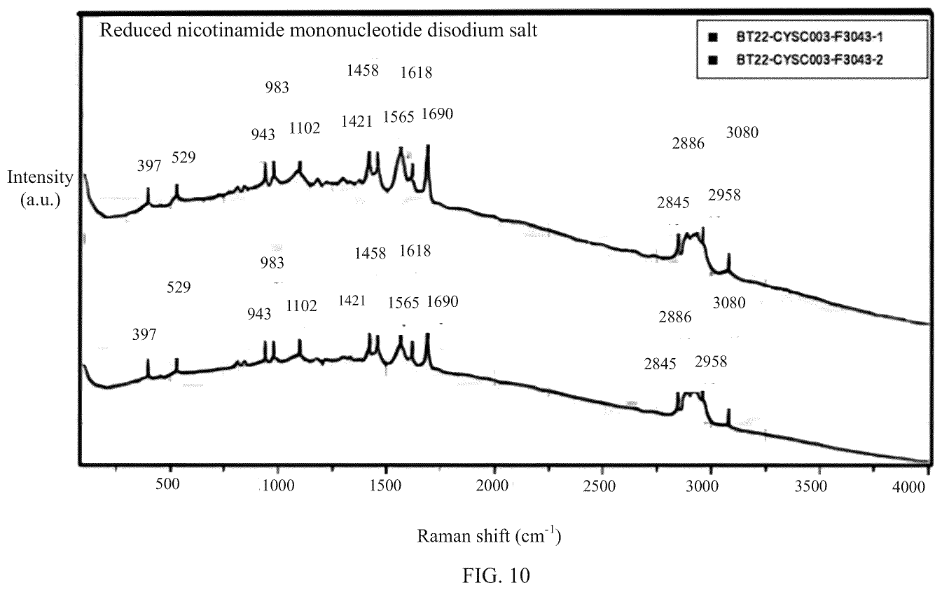

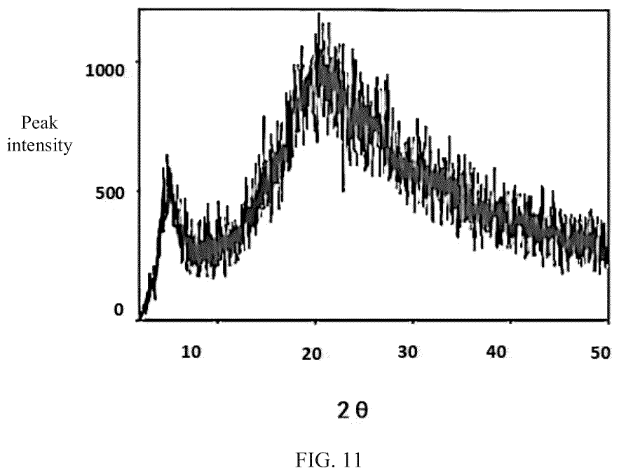

Preparation of a freeze-dried powder: The concentrated aqueous solution of the reduced β-nicotinamide mononucleotide disodium salt was fed into a 2 m² vacuum freeze dryer for freeze-drying, then pre-frozen at −50 °C for 2 hours, and gradually heated to 30 °C for 72 hours to obtain 1.71 kg of an amorphous pine needle-like light yellow powder, that is, an amorphous freeze-dried powder of the reduced β-nicotinamide mononucleotide disodium salt, being fluffy like cotton fluff. The freeze-dried powder of the reduced β-nicotinamide mononucleotide disodium salt remaining on a drying tray becomes viscous after exposure to the air for half an hour and becomes an oily substance after one hour. A weight loss on drying of the freeze-dried powder of the reduced β-nicotinamide mononucleotide disodium salt at 120 °C is 5.89%, and a purity detected by a high performance liquid chromatograph (with a C18 chromatographic column, a mobile phase of water/methanol, and a detection wavelength of 340 nm) is 99.18%. A sodium ion content detected by an ion chromatograph is 11.41%, and the sodium ion content is calculated to be 12.12% after deducting moisture. The obtained freeze-dried powder does not have a pungent ammonia odor, and the sodium ion content is close to a theoretical disodium salt content standard of 12.11%. A thermogravimetric analysis spectrogram of the obtained freeze-dried powder of the reduced β-nicotinamide mononucleotide disodium salt is shown in FIG. 6, a differential scanning calorimetry spectrogram is shown in FIG. 8, a Raman spectrogram is shown in FIG. 10, and an X-ray powder diffraction pattern is shown in FIG. 11. The X-ray powder diffraction pattern shows that obvious amorphous characteristic peaks exist, that is, no sharp absorption peaks exist, which is a diffuse pattern. The differential scanning calorimetry spectrogram shows that the obtained freeze-dried powder has two characteristic glass transition temperatures in temperature ranges of 50.92 °C-56.94 °C and 80 °C-110 °C, with peak values of the two glass transition temperatures being 53.99 °C and 94.30 °C, respectively. The obtained freeze-dried powder has a characteristic melting point temperature in a temperature range of 201.37 °C-236.92 °C, and further, a peak value of the characteristic melting point temperature is 224.36 °C The obtained freeze-dried powder has stronger characteristic peaks at Raman shifts (cm⁻¹) of 942 cm⁻¹ and 983 cm⁻¹ in the Raman spectrogram.

### Example 3 Preparation of an Amorphous Compound of a Reduced β-Nicotinamide Mononucleotide Disodium Salt (with the Above Step (3) Omitted and a Product Obtained by a Crystallization Method)

Preparation of an aqueous solution of a reduced β-nicotinamide mononucleotide disodium salt: 15 L of pure water was added into a 50 L glass reactor and heated to 39 °C, 1.49 kg of NMN was added into the glass reactor, and under stirring conditions, a pH was adjusted to 9.5-10.0 with a 10 mol/L sodium hydroxide solution until the resulting solution was dissolved and clarified. 0.78 kg of thiourea dioxide was added into the glass reactor to carry out a reaction at a temperature controlled at 39 °C-44 °C under stirring for 7 hours. During the reaction, the pH was adjusted with a 10 mol/L sodium hydroxide solution, wherein the pH was controlled at 9.5-10.5. After completion of the reaction, the pH was adjusted to 10.5-11.0 with a 10 mol/L sodium hydroxide solution. A preparative high performance liquid chromatograph (a diameter of a chromatographic column was 15 cm, and a column volume was 6 L; a packing material was a polystyrene reversed-phase packing material, with a packing material model of Unis P40-300, provided by NanoMicro Technology Co., Ltd.; mobile phases were water and ethanol; a loading volume of a reaction solution each time was 1.5 L-2.0 L, and a product was eluted with pure water after loading; fractions with a purity of greater than 98.50% were collected in sections to serve as qualified products and sent to the next process step, and fractions with a purity of less than 98.50% were collected to serve as unqualified products and sent to the reaction solution for re-purification, with a time consumption of 25-30 minutes for each purification; and after completion of the purification for 10 times, the reaction solution was eluted with a 20% ethanol aqueous solution to remove impurities on the chromatographic column, the chromatographic column was rinsed with pure water, and then the reaction solution was continuously loaded for purification, with a wavelength of an ultraviolet detector being 340 nm) was used for purification and separation to obtain an aqueous solution of reduced β-nicotinamide mononucleotide. Nanofiltration and concentration were performed with a nanofiltration membrane to 12 L, and during the nanofiltration and the concentration, the pH value was adjusted with a 10 mol/L sodium hydroxide solution and maintained at 10.5-11.0 to obtain a concentrated aqueous solution of a reduced β-nicotinamide mononucleotide disodium salt, which contained 1.52 kg of the reduced β-nicotinamide mononucleotide disodium salt as detected by a high performance liquid chromatograph (with a C18 chromatographic column, a detection wavelength of 340 nm, an external standard method for detection, and a standard product purchased from Hubei WeiShi Chemical Reagent Co., Ltd.).

Preparation of an amorphous compound of the reduced β-nicotinamide mononucleotide disodium salt: The concentrated aqueous solution of the reduced β-nicotinamide mononucleotide disodium salt was added into a 50 L glass reactor, 24 L of ethanol was added at room temperature (20 °C-25 °C) (unless otherwise specified below, the ethanol and a poor solvent both referred to anhydrous ethanol), and stirring was performed continuously for 2 hours until the solution was still clarified. Under stirring conditions, 0.74 kg of xylose was added, stirring was performed continuously at room temperature (20 °C-25 °C) for 0.5 hour until the solution was clarified and an oily substance appeared at the bottom, and stirring was performed continuously at room temperature (20 °C-25 °C) for 2.5 hours until the oily substance at the bottom was gradually increased and no precipitate separated out. Under stirring conditions, 12 L of ethanol was continuously added dropwise at a dropping rate of 3.0 L/h. After completion of the dropping, no precipitate separated out. Stirring was performed continuously at room temperature (20 °C-25 °C) for 2 hours until a large amount of an oily substance was generated at the bottom of the reactor and no precipitate separated out. Under stirring conditions, cooling was performed to 10 °C-15 °C, and stirring was performed for 2 hours until no precipitate separated out. Under stirring conditions, cooling was performed to 0 °C-5 °C, and stirring was performed for 2 hours until no precipitate separated out. Preparation of the amorphous compound of the reduced β-nicotinamide mononucleotide disodium salt failed.

### Example 4 Preparation of an Amorphous Compound of a Reduced β-Nicotinamide Mononucleotide Disodium Salt

Preparation of an aqueous solution of a reduced β-nicotinamide mononucleotide disodium salt: 30 L of pure water was added into a 50 L glass reactor and heated to 42 °C, 3.12 kg of NMN was added into the glass reactor, and under stirring conditions, a pH was adjusted to 9.5-10.5 with a 10 mol/L sodium hydroxide solution until the resulting solution was dissolved and clarified. 1.72 kg of thiourea dioxide was added into the glass reactor to carry out a reaction at a temperature controlled at 39 °C-44 °C under stirring for 7 hours. During the reaction, the pH was adjusted with a 10 mol/L sodium hydroxide solution, wherein the pH was controlled at 9.5-10.5. After completion of the reaction, the pH was adjusted to 10.5-11.0 with a 10 mol/L sodium hydroxide solution. A preparative high performance liquid chromatograph (a diameter of a chromatographic column was 15 cm, and a column volume was 6 L; a packing material was a polystyrene reversed-phase packing material, with a packing material model of Unis P40-300, provided by NanoMicro Technology Co., Ltd.; mobile phases were water and ethanol; a loading volume of a reaction solution each time was 1.5 L-2.0 L, and a product was eluted with pure water after loading; fractions with a purity of greater than 98.50% were collected in sections to serve as qualified products and sent to the next process step, and fractions with a purity of less than 98.50% were collected to serve as unqualified products and sent to the reaction solution for re-purification, with a time consumption of 25-30 minutes for each purification; and after completion of the purification for 10 times, the reaction solution was eluted with a 20% ethanol aqueous solution to remove impurities on the chromatographic column, the chromatographic column was rinsed with pure water, and then the reaction solution was continuously loaded for purification, with a wavelength of an ultraviolet detector being 340 nm) was used for purification and separation to obtain an aqueous solution of reduced β-nicotinamide mononucleotide. The obtained aqueous solution of the reduced β-nicotinamide mononucleotide was subjected to ammonia removal and purification operation using a 001X7 strongly acidic cation exchange resin (a resin volume was 12 L, the solution was treated with a 0.5-1 mol/L sodium hydroxide solution before the purification to obtain a sodium form and then rinsed with purified water to a pH of 7-9 to load the aqueous solution of the reduced β-nicotinamide mononucleotide, and the loaded solution was eluted with pure water at a flow rate of 1-1.5 times column volume/hour). After the ammonia removal and the purification using the 001X7 strongly acidic cation exchange resin, a purified solution of the reduced β-nicotinamide mononucleotide was obtained. Nanofiltration and concentration were performed with a nanofiltration membrane to 36 L, and during the nanofiltration and the concentration, the pH value was adjusted with a 10 mol/L sodium hydroxide solution and maintained at 10.5-11.0 to obtain a concentrated aqueous solution of a reduced β-nicotinamide mononucleotide disodium salt, which contained 3.24 kg of the reduced β-nicotinamide mononucleotide disodium salt as detected by a high performance liquid chromatograph (with a C18 chromatographic column, a detection wavelength of 340 nm, an external standard method for detection, and a standard product purchased from Hubei WeiShi Chemical Reagent Co., Ltd.).

Preparation of an amorphous compound of the reduced β-nicotinamide mononucleotide disodium salt: 6 L of the concentrated aqueous solution of the reduced β-nicotinamide mononucleotide disodium salt obtained in the above step (containing 0.53 kg of the reduced β-nicotinamide mononucleotide disodium salt as detected by a high performance liquid chromatograph) was weighed and added into a 50 L glass reactor, 12 L of ethanol was added at room temperature (20 °C-25 °C), and stirring was performed continuously for 2 hours until the solution was still clarified. Under stirring conditions, 0.74 kg of xylose was added, stirring was performed continuously at room temperature (20 °C-25 °C) for 0.5 hour until the solution was clarified and a layer of an oily substance appeared at the bottom, a part of the oily substance at the bottom was gradually precipitated out as a powder, and a precipitate was suspended in the solution. Stirring was performed continuously at room temperature (20 °C-25 °C) for 2.5 hours until a large amount of a precipitate separated out, the solution became turbid, and a part of the precipitate aggregated into a lump. Under stirring conditions, 12 L of ethanol was continuously added dropwise at a dropping rate of 3.0 L/h. After completion of the dropping, stirring was performed continuously at room temperature (20 °C-25 °C) for 2 hours. As detected by a high performance liquid chromatograph, 0.24 kg of the reduced β-nicotinamide mononucleotide disodium salt remained in a supernatant of a crystallization mother solution. Under stirring conditions, 12 L of ethanol was continuously added dropwise at a dropping rate of 3.0 L/h. After completion of the dropping, stirring was performed continuously at room temperature (20 °C-25 °C) for 2 hours. As detected by a high performance liquid chromatograph, 0.12 kg of the reduced β-nicotinamide mononucleotide disodium salt remained in the supernatant of the crystallization mother solution. Under stirring conditions, 6 L of ethanol was continuously added dropwise at a dropping rate of 3.0 L/h. After completion of the dropping, stirring was performed continuously at room temperature (20 °C-25 °C) for 2 hours. As detected by a high performance liquid chromatograph, 0.068 kg of the reduced β-nicotinamide mononucleotide disodium salt remained in the supernatant of the crystallization mother solution. After discharging and filtration, a filter cake was washed with 5 L of ethanol and vacuum dried in a vacuum drying oven at 30 °C for 15 hours to obtain 0.45 kg of a grayish-white dried solid powder. A freeze-dried powder of the reduced β-nicotinamide mononucleotide disodium salt remaining on a drying tray remains in a powder state after exposure to the air for half an hour, still remains in the powder state after one hour, has no pungent ammonia odor, and compared with the freeze-dried powder of the reduced β-nicotinamide mononucleotide disodium salt prepared and obtained in Example 2, has better flowability, appearance, color, and luster. A weight loss on drying at 120 °C is 2.59%, and a purity detected by a high performance liquid chromatograph (with a C18 chromatographic column, a mobile phase of water/methanol, and a detection wavelength of 340 nm) is 98.89%. A sodium ion content detected by an ion chromatograph is 11.77%, and the sodium ion content is calculated to be 12.08% after deducting moisture. The obtained freeze-dried powder does not have a pungent ammonia odor, and the sodium ion content is close to a theoretical disodium salt content standard of 12.11%, that is, the obtained grayish-white dried solid powder is the amorphous compound of the reduced β-nicotinamide mononucleotide disodium salt.

### Example 5 Preparation of an Amorphous Compound of a Reduced β-Nicotinamide Mononucleotide Disodium Salt

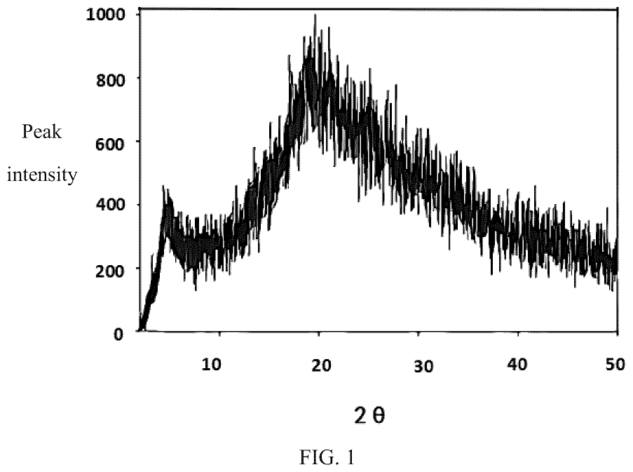

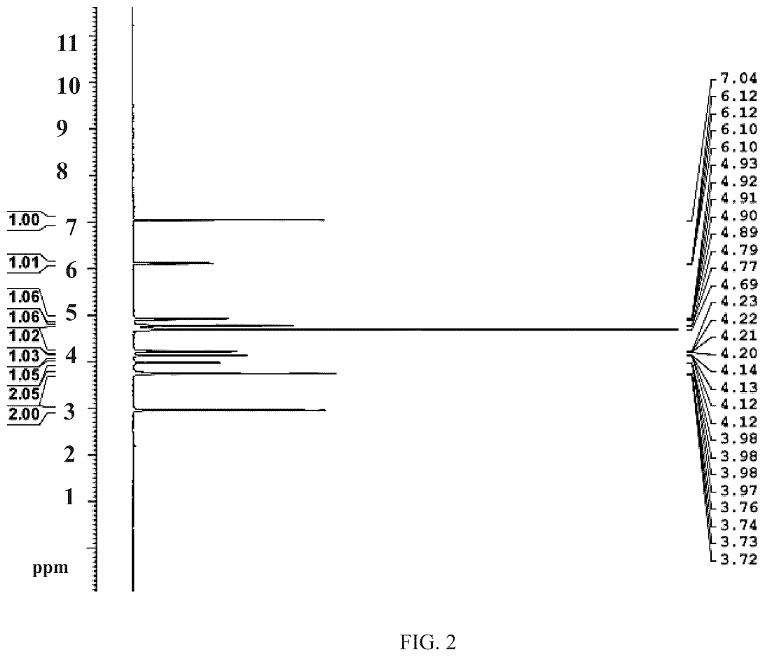

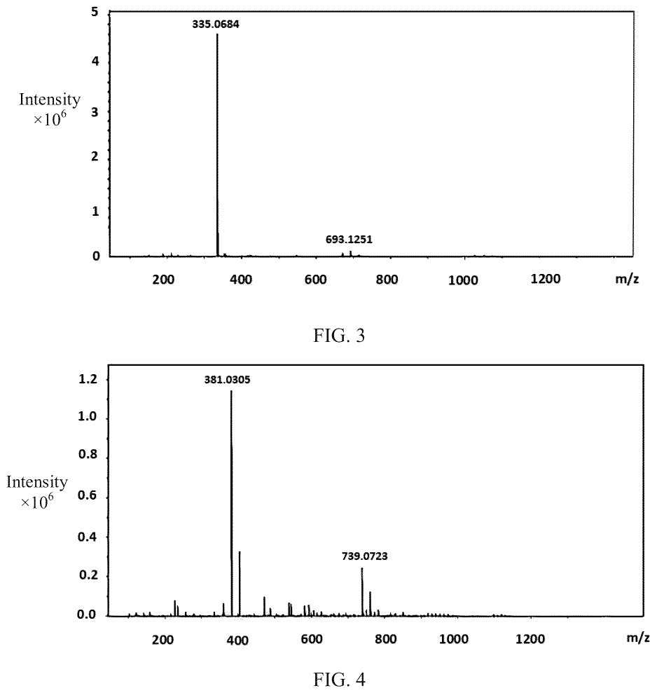

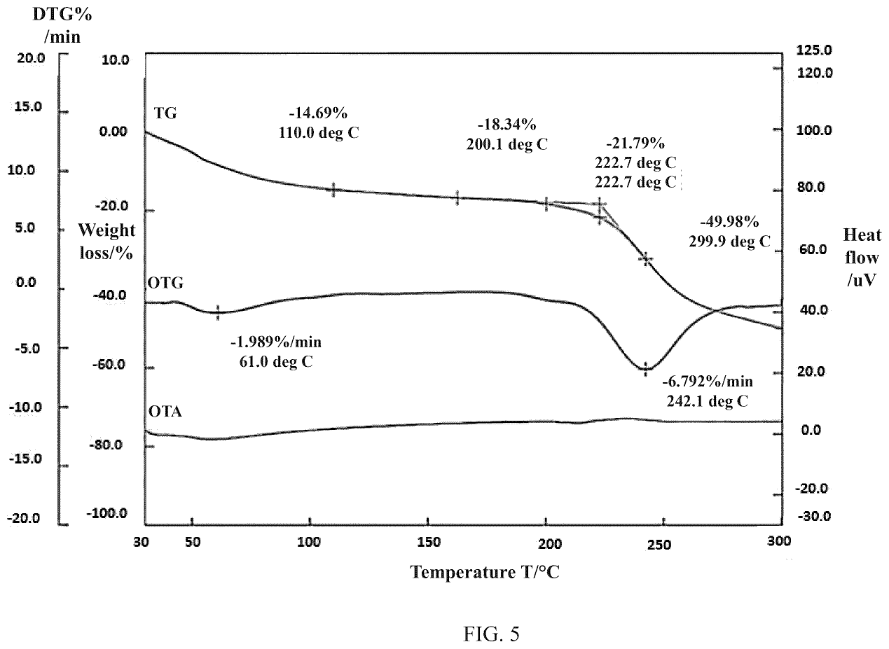

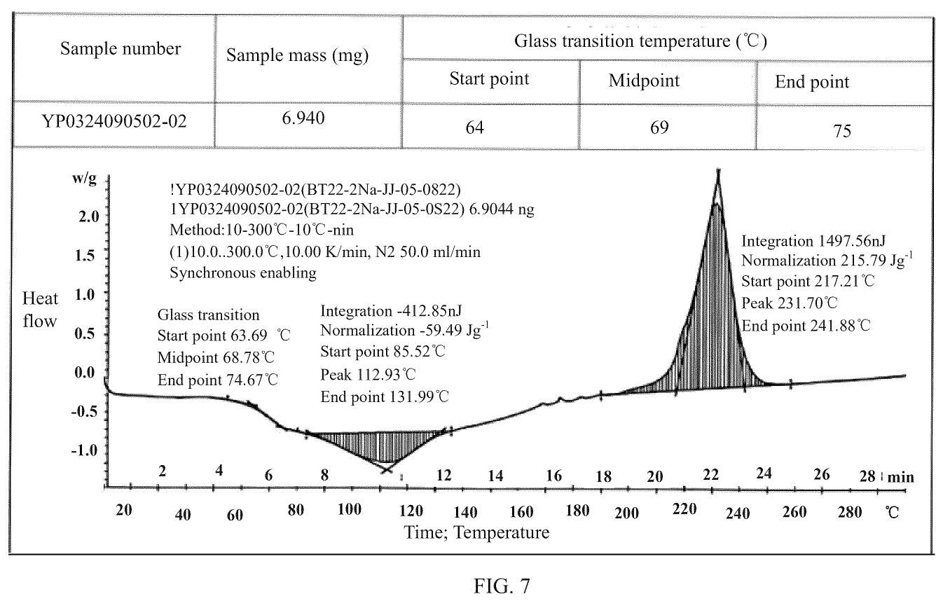

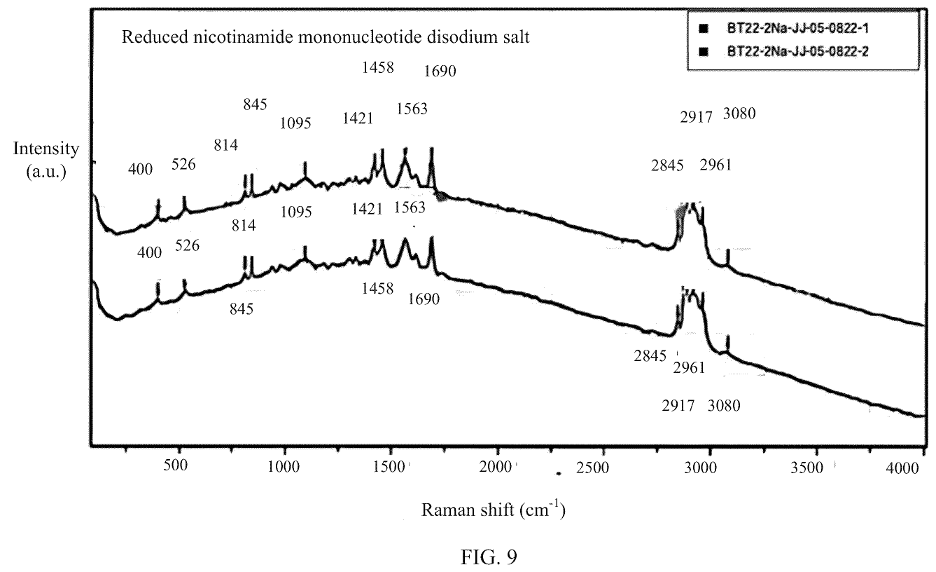

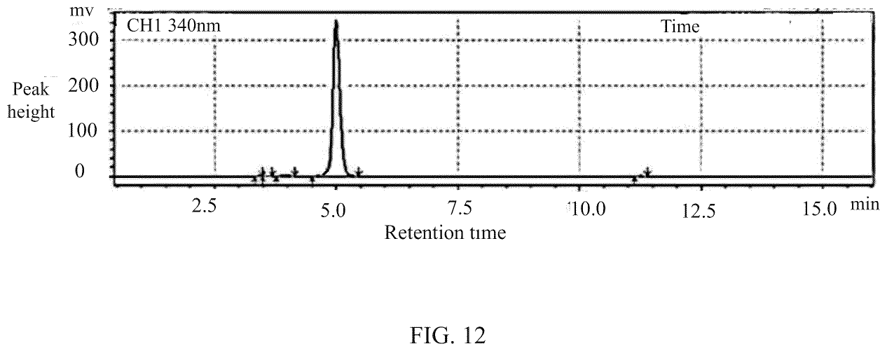

Preparation of an amorphous compound of a reduced β-nicotinamide mononucleotide disodium salt: 6 L of the concentrated aqueous solution of the reduced β-nicotinamide mononucleotide disodium salt obtained in Example 4 (containing 0.54 kg of the reduced β-nicotinamide mononucleotide disodium salt as detected by a high performance liquid chromatograph) was weighed and added into a 50 L glass reactor, 12 L of ethanol was added at room temperature (20 °C-25 °C), and stirring was performed continuously for 2 hours until the solution was still clarified. Under stirring conditions, 0.02 kg of the amorphous compound of the reduced β-nicotinamide mononucleotide disodium salt obtained in Example 4 was added, and stirring was performed continuously at room temperature (20 °C-25 °C) for 0.5 hour until a precipitate was suspended in the solution. Stirring was performed continuously at room temperature (20 °C-25 °C) for 2.5 hours until a large amount of a precipitate separated out, the solution became turbid, and no agglomeration phenomenon was observed. Under stirring conditions, cooling was performed to 10 °C-15 °C, and 12 L of ethanol was continuously added dropwise at a dropping rate of 4.0 L/h. After completion of the dropping, stirring was performed continuously at a low temperature (10 °C-15 °C) for 2 hours. As detected by a high performance liquid chromatograph, 0.18 kg of the reduced β-nicotinamide mononucleotide disodium salt remained in a supernatant of a crystallization mother solution. Under stirring conditions at a low temperature (10 °C-15 °C), 12 L of ethanol was continuously added dropwise at a dropping rate of 4.0 L/h. After completion of the dropping, stirring was performed continuously at a low temperature (10 °C-15 °C) for 2 hours. As detected by a high performance liquid chromatograph, 0.086 kg of the reduced β-nicotinamide mononucleotide disodium salt remained in the supernatant of the crystallization mother solution. Under stirring conditions at a low temperature (10 °C-15 °C), 6 L of ethanol was continuously added dropwise at a dropping rate of 4.0 L/h. After completion of the dropping, stirring was performed continuously at a low temperature (10 °C-15 °C) for 2 hours. As detected by a high performance liquid chromatograph, 0.044 kg of the reduced β-nicotinamide mononucleotide disodium salt remained in the supernatant of the crystallization mother solution. After discharging and filtration, a filter cake was washed with 5 L of ethanol and vacuum dried in a vacuum drying oven at 25 °C for 24 hours to obtain 0.48 kg of a grayish-white dried solid powder. A freeze-dried powder of the reduced β-nicotinamide mononucleotide disodium salt remaining on a drying tray remains in a powder state after exposure to the air for half an hour, still remains in the powder state after one hour, has no pungent ammonia odor, and compared with the amorphous compound of the reduced β-nicotinamide mononucleotide disodium salt prepared and obtained in Example 4, has consistent flowability, appearance, color, and luster, without agglomeration phenomenon. A weight loss on drying at 120 °C is 3.70%, and a purity detected by a high performance liquid chromatograph (with a C18 chromatographic column, a mobile phase of water/methanol, and a detection wavelength of 340 nm) is 99.53%. Compared with Example 4, the obtained solid powder has an obviously increased purity and yield. A sodium ion content detected by an ion chromatograph is 11.66%, and the sodium ion content is calculated to be 12.11% after deducting moisture. The obtained freeze-dried powder does not have a pungent ammonia odor, and the sodium ion content meets a theoretical disodium salt content standard of 12.11%, that is, the obtained grayish-white dried solid powder is the amorphous compound of the reduced β-nicotinamide mononucleotide disodium salt. An X-ray powder diffraction pattern of the obtained amorphous compound of the reduced β-nicotinamide mononucleotide disodium salt is shown in FIG. 1. The X-ray powder diffraction pattern shows that obvious amorphous characteristic peaks exist, that is, no sharp absorption peaks exist, which is a diffuse pattern, proving that the obtained solid is the amorphous compound of the reduced β-nicotinamide mononucleotide disodium salt. A nuclear magnetic resonance hydrogen spectrogram is shown in FIG. 2, a negative-mode mass spectrogram is shown in FIG. 3, a positive-mode mass spectrogram is shown in FIG. 4, a thermogravimetric analysis spectrogram is shown in FIG. 5, a differential scanning calorimetry spectrogram is shown in FIG. 7, a Raman spectrogram is shown in FIG. 9, and an HPLC detection chromatogram is shown in FIG. 12. The obtained amorphous compound of the reduced β-nicotinamide mononucleotide disodium salt is characterized in that the amorphous compound has two characteristic glass transition temperatures in temperature ranges of 63.69 °C-74.67 °C and 85.52 °C-131.99 °C in the differential scanning calorimetry spectrogram, and further, peak values of the two glass transition temperatures are 68.78 °C and 112.93 °C, respectively. The obtained amorphous compound has a characteristic melting point temperature in a temperature range of 217.21 °C-241.88 °C, with a peak value of the characteristic melting point temperature being 231.70 °C The obtained amorphous compound has stronger characteristic peaks at Raman shifts (cm⁻¹) of 814 cm⁻¹ and 845 cm⁻¹ in the Raman spectrogram.

### Example 6 Preparation of an Amorphous Compound of a Reduced β-Nicotinamide Mononucleotide Disodium Salt

Preparation of an amorphous compound of a reduced β-nicotinamide mononucleotide disodium salt: 6 L of the concentrated aqueous solution of the reduced β-nicotinamide mononucleotide disodium salt obtained in Example 4 (containing 0.52 kg of the reduced β-nicotinamide mononucleotide disodium salt as detected by a high performance liquid chromatograph) was weighed and added into a 50 L glass reactor, 12 L of ethanol was added at room temperature (20 °C-25 °C), and stirring was performed continuously for 2 hours until the solution was still clarified. Under stirring conditions, 0.02 kg of the amorphous compound of the reduced β-nicotinamide mononucleotide disodium salt obtained in Example 5 was added, and stirring was performed continuously at room temperature (20 °C-25 °C) for 0.5 hour until a precipitate was suspended in the solution. Stirring was performed continuously at room temperature (20 °C-25 °C) for 2.5 hours until a large amount of a precipitate separated out, the solution became turbid, and no agglomeration phenomenon was observed. Under stirring conditions, cooling was performed to 0 °C-5 °C, and 12 L of ethanol was continuously added dropwise at a dropping rate of 6.0 L/h. After completion of the dropping, stirring was performed continuously at a low temperature (0 °C-5 °C) for 2 hours. As detected by a high performance liquid chromatograph, 0.14 kg of the reduced β-nicotinamide mononucleotide disodium salt remained in a supernatant of a crystallization mother solution. Under stirring conditions at a low temperature (0 °C-5 °C), 12 L of ethanol was continuously added dropwise at a dropping rate of 6.0 L/h. After completion of the dropping, stirring was performed continuously at a low temperature (0 °C-5 °C) for 2 hours. As detected by a high performance liquid chromatograph, 0.0686 kg of the reduced β-nicotinamide mononucleotide disodium salt remained in the supernatant of the crystallization mother solution. Under stirring conditions at a low temperature (10 °C-15 °C), 6 L of ethanol was continuously added dropwise at a dropping rate of 6.0 L/h. After completion of the dropping, stirring was performed continuously at a low temperature (0 °C-5 °C) for 2 hours. As detected by a high performance liquid chromatograph, 0.035 kg of the reduced β-nicotinamide mononucleotide disodium salt remained in the supernatant of the crystallization mother solution. After discharging and filtration, a filter cake was washed with 5 L of ethanol and vacuum dried in a vacuum drying oven at 25 °C for 24 hours to obtain 0.49 kg of a grayish-white dried solid powder. A freeze-dried powder of the reduced β-nicotinamide mononucleotide disodium salt remaining on a drying tray remains in a powder state after exposure to the air for half an hour, still remains in the powder state after one hour, has no pungent ammonia odor, and compared with the amorphous compound of the reduced β-nicotinamide mononucleotide disodium salt prepared and obtained in Example 5, has consistent flowability, appearance, color, and luster, without agglomeration phenomenon. A weight loss on drying at 120 °C is 3.58%, and a purity detected by a high performance liquid chromatograph (with a C18 chromatographic column, a mobile phase of water/methanol, and a detection wavelength of 340 nm) is 99.44%. Compared with Example 5, the obtained solid powder has a decreased purity and an increased yield. A sodium ion content detected by an ion chromatograph is 11.67%, and the sodium ion content is calculated to be 12.10% after deducting moisture. The obtained freeze-dried powder does not have a pungent ammonia odor, and the sodium ion content meets a theoretical disodium salt content standard of 12.11%, that is, the obtained grayish-white dried solid powder is the amorphous compound of the reduced β-nicotinamide mononucleotide disodium salt.

### Performance Analysis of Products of Various Examples:

### Detection of the Reduced β-Nicotinamide Mononucleotide Disodium Salts Obtained in Examples 1 to 6:

The reduced β-nicotinamide mononucleotide disodium salt has a theoretical sodium ion content value of 12.105%, a molecular weight of 380, and a sodium-free molecular weight of 336. The solids obtained in Examples 1 to 6 were respectively subjected to sodium ion content detection (see various examples for details). The results show that among the products obtained in Examples 1 to 6, except for Example 1, the sodium ion contents of the solid powders after deducting moisture are 12.08%-12.12% (the sodium ion content in Example 1 has a larger difference from the theoretical value due to an ammonia residue), which conform to the characterization of the reduced β-nicotinamide mononucleotide disodium salt. Further, the solid obtained in Example 5 is subjected to nuclear magnetic resonance and hydrogen spectrometry detection, and high-resolution positive and negative mass spectrometry detection (the XRPD pattern is shown in FIG. 1, the hydrogen spectrogram is shown in FIG. 2, the negative-mode mass spectrogram is shown in FIG. 3, and the positive-mode mass spectrogram is shown in FIG. 4). The obtained solid has a molecular ion peak at M+: 381.0305 in the high-resolution positive-mode mass spectrogram, which is consistent with the molecular weight of the reduced β-nicotinamide mononucleotide disodium salt; and the obtained solid has a molecular ion peak at M−: 335.0684 in the high-resolution negative-mode mass spectrogram, which is consistent with the sodium-free molecular weight, being 336, of the reduced β-nicotinamide mononucleotide disodium salt, proving that the obtained solid is the reduced β-nicotinamide mononucleotide disodium salt. A structural formula is as follows:

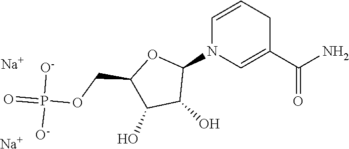 [SMILES: [Na+] ; NC(=O)C1=CN([C@@H]2O[C@H](COP(=O)([O-])[O-])[C@@H](O)[C@H]2O)C=CC1 ; [Na+]]

During the purification in Examples 1 and 2, after the step (3) is added, the residual ammonia odor is effectively removed from the product, thereby avoiding potential health and safety risks during product applications.

During the crystallization in Examples 4 to 6, after adding the poor solvent and stirring for 2 hours, the solution is still clarified, showing that the reduced β-nicotinamide mononucleotide disodium salt is difficult to precipitate without the addition of the seed crystal. After the seed crystal is added, stirring is performed for a certain period at a maintained temperature, and a precipitate begins to separate out. After the precipitate separates out, the poor solvent is continuously added dropwise to continuously precipitate the reduced β-nicotinamide mononucleotide disodium salt, so that the product yield is constantly increased. Meanwhile, at the stage of adding the poor solvent dropwise, different temperatures have an obvious impact on the product yield and the product purity. The results of Examples 4 to 6 show that the optimal temperature range is 10 °C-15 °C, which not only ensures a higher yield, but also obtains a highest product purity, and is more favorable for industrial production.

Through the comparison between the product purity, flowability, hygroscopicity, and room-temperature stability (as reflected in subsequent stability investigations) of the products of Example 5 and Example 2, the amorphous compound of the reduced β-nicotinamide mononucleotide disodium salt obtained in Example 5 has a higher purity, better flowability, lower hygroscopicity, and better room-temperature stability. The compound has a wider application prospect in fields such as health care products, cosmetics, and food additives.

### Comparison of Differential Scanning Calorimetry and Thermogravimetric Analysis

Different amorphous forms of amorphous compounds have unique physical, thermodynamic, and kinetic characteristics, and unique thermodynamic properties or weight loss percentages of the amorphous compounds can be detected by DSC and TGA.

Through the comparison between the DSC analysis of Example 5 and Example 2, the amorphous product of Example 5 has a higher melting point temperature and a higher glass transition temperature. The higher melting point and the higher glass transition temperature indicate that the product has higher thermal stability.

Through the comparative analysis of the products of Example 5 and Example 2, thermogravimetry (TG) curves show that the product of Example 5 has a more obvious curve platform area, and derivative thermogravimetry (DTG) curves show that the product of Example 5 has a higher temperature at the maximum weight loss rate. Generally, the higher temperature at the maximum weight loss rate indicates that a sample has a higher thermal decomposition temperature and better thermal stability.

### Comparative Analysis of Raman Spectroscopy:

Since amorphous compounds exhibit diffusion peaks in powder X-ray diffraction (PXRD) and lack sharp diffraction peaks, traditional PXRD technology has a certain difficulty in the identification of amorphous forms. A Raman spectroscopy technology utilizes differences in vibrational-rotational transition energy levels of certain chemical bonds or changes in molecular polarizability in amorphous compound molecules to achieve identification based on differences in the frequency, peak shape, peak position, and peak intensity of a characteristic spectrum, which is conducive to identifying the amorphous forms of the amorphous compounds more accurately. Through the comparison between Raman spectra (FIGS. 9 and 10), it can be seen that the amorphous compound obtained by crystallization in Example 5 of the present disclosure and the amorphous compound obtained by freeze-drying in Example 2 have obvious differences in shift and intensity of the Raman spectra, indicating that the two amorphous compounds have different amorphous forms. This distinction directly affects the stability, hygroscopicity, and other properties of the amorphous compounds.

A Raman spectroscopy test is performed by scanning using a laser confocal Raman spectrometer at an excitation wavelength of 532 nm, a grating of 600 lines, an objective lens of 50×, an integration time of 5 seconds, and an accumulation number of times of 3 times.

Through the comparison between the Raman spectra of the products of Example 5 and Example 2, it can be seen that both the shift and intensity of characteristic peaks of the two are different, indicating that the two exist in different amorphous forms.

### Stability Investigation I:

The amorphous compound of the reduced β-nicotinamide mononucleotide disodium salt prepared and obtained in Example 5 of the present disclosure and the amorphous freeze-dried powder obtained in Comparative Example 2 were respectively sealed and placed in a self-sealing bag to investigate the stability under room-temperature conditions of 25 °C-35 °C and 65%-85% RH. Stability investigation results shown in the table below are obtained.

| | Moisture content % | | NMNH purity (HPLC, | | | |
|---|---|---|---|---|---|---|
| Time/ | Example | Example | area %) | | Appearance state | |
| day | 2 | 5 | Example 2 | Example 5 | Example 2 | Example 5 |
| 0 | 5.89 | 3.70 | 99.18 | 99.53 | Light yellow | Off-white powder |
| | | | | | needle-like powder | |
| 1 | 5.87 | 3.68 | 99.19 | 99.51 | Light yellow | Off-white powder |
| | | | | | needle-like powder | |
| 5 | 7.82 | 3.74 | 99.15 | 99.50 | Light yellow | Off-white powder |
| | | | | | needle-like powder | |
| 10 | 10.18 | 3.92 | 98.99 | 99.51 | Light yellow | Off-white powder |
| | | | | | needle-like powder | |
| 20 | 14.81 | 4.32 | 98.64 | 99.48 | Light yellow | Off-white powder |
| | | | | | needle-like powder | |
| 30 | 19.38 | 5.10 | 98.15 | 99.45 | Light yellow | Off-white powder |
| | | | | | needle-like powder | |
| 45 | 26.88 | 6.12 | 96.04 | 99.37 | Reddish-brown | Off-white powder |
| | | | | | powder | |
| 60 | 30.18 | 7.05 | 90.14 | 99.18 | Reddish-brown | Off-white powder |
| | | | | | powder | |
| 90 | 36.62 | 8.12 | 61.34 | 99.01 | Black gelatinous | Off-white powder |
| | | | | | substance | |
| 120 | / | 9.88 | 0 | 98.66 | Black oily | Off-white powder |
| | | | (completely | (accelerated | substance | |
| | | | degraded) | degradation) | | |

It can be seen from the table above that the room-temperature stability of the amorphous compound of the reduced β-nicotinamide mononucleotide disodium salt prepared and obtained in Example 5 of the present disclosure is obviously better than that of the amorphous freeze-dried powder of the reduced nicotinamide mononucleotide disodium salt prepared and obtained in Example 2 of the present disclosure, and after sealing and placing in the self-sealing bag under the room-temperature conditions of 25 °C-35 °C and 65%-85% RH within 3 months, the purity is decreased by less than 1%, indicating significantly increased stability. Meanwhile, it can be seen from the table above that a moisture residue in the obtained solid powder of the reduced nicotinamide mononucleotide disodium salt has an obvious impact on the room-temperature stability of products. When the moisture residue exceeds 10%, degradation is obviously accelerated. The room-temperature stability of the amorphous compound of the reduced β-nicotinamide mononucleotide disodium salt prepared and obtained in Example 5 of the present disclosure is obviously better than that of the amorphous freeze-dried powder of the reduced nicotinamide mononucleotide disodium salt obtained in Example 2. According to a moisture increase rate during the placement at room temperature, the moisture increase rate of the amorphous freeze-dried powder of the reduced nicotinamide mononucleotide disodium salt obtained in Example 2 is obviously higher than that of the amorphous compound of the reduced β-nicotinamide mononucleotide disodium salt obtained in Example 5. Moreover, the amorphous compound of the reduced β-nicotinamide mononucleotide disodium salt obtained in Example 5 has a lower initial moisture residue.

### Stability Investigation II:

The amorphous compound of the reduced β-nicotinamide mononucleotide disodium salt prepared and obtained in Example 5 and the amorphous freeze-dried powder obtained in Example 2 were respectively placed in a constant temperature and humidity chamber under open conditions at 25 °C and 65% RH to investigate the stability under room-temperature conditions. Stability investigation results shown in the table below are obtained.

| | | | NMNH purity (HPLC, | | | |
|---|---|---|---|---|---|---|
| | Moisture content % | | area %) | | Appearance state | |
| Time/day | Example 2 | Example 5 | Example 2 | Example 5 | Example 2 | Example 5 |
| 0 | 5.89 | 3.70 | 99.18 | 99.53 | Light yellow | Off-white |
| | | | | | needle-like | powder |
| | | | | | powder | |
| 0.5 | 41 . . . 87 | 12.57 | 95.19 | 98.41 | Reddish-brown | Light yellow |
| | | | | | gelatinous | powder |
| | | | | | substance | |
| 1 | / | 25.14 | 75 | 94.61 | Reddish-brown | Yellow powder |
| | | | | | oily substance | |
| 2 | / | 36.28 | 28.24 | 88.49 | Black oily | Reddish-brown |
| | | | | | substance | powder |

It can be seen from the table above that the room-temperature stability of the amorphous compound of the reduced β-nicotinamide mononucleotide disodium salt of Example 5 is obviously better than that of the amorphous freeze-dried powder of the reduced nicotinamide mononucleotide disodium salt of Example 2 after placement under open conditions. Meanwhile, it can be seen from the table above that a moisture residue has an obvious impact on the room-temperature stability of products. The room-temperature stability of the amorphous compound of the reduced β-nicotinamide mononucleotide disodium salt obtained in Example 5 is obviously better than that of the amorphous freeze-dried powder of the reduced nicotinamide mononucleotide disodium salt obtained in Example 2. According to a moisture increase rate during the placement at room temperature under open conditions, the moisture increase rate of the amorphous freeze-dried powder of the reduced nicotinamide mononucleotide disodium salt prepared and obtained in Example 2 is obviously higher than that of the amorphous compound of the reduced β-nicotinamide mononucleotide disodium salt prepared and obtained in Example 5. Meanwhile, the obtained solid powder of the reduced nicotinamide mononucleotide disodium salt exhibits varying degrees of color deepening, accompanied by rapid degradation, after exposure to the air, inferring that the situation may be caused by a 1,4-dihydropyridine ring in the structure of the reduced β-nicotinamide mononucleotide disodium salt, which has a higher reducing activity and is prone to undergoing a redox reaction with oxygen in the air under high humidity conditions, leading to degradation. Thus, during long-term storage conditions of the reduced β-nicotinamide mononucleotide disodium salt, the humidity needs to be controlled, and isolation from the air needs to be performed as much as possible.

Finally, it is to be noted that the above preferred examples are used only to illustrate the technical solutions of the present disclosure, rather than to limit the present disclosure. Although the present disclosure has been described in detail through the above preferred examples, it is understood by those skilled in the art that various changes in forms and details may be made without deviating from the scope defined by the claims of the present disclosure.

What is claimed is:

1. A preparation method for an amorphous compound of a reduced β-nicotinamide mononucleotide disodium salt, comprising the following steps:
(1) using β-nicotinamide mononucleotide as a raw material, thiourea dioxide as a reducing agent, and pure water as a reaction solvent to carry out a reaction under alkaline conditions to obtain an aqueous solution of reduced β-nicotinamide mononucleotide;
(2) separating and purifying the aqueous solution of the reduced β-nicotinamide mononucleotide using a preparative high performance liquid chromatograph;
(3) removing an ammonium salt using a strongly acidic cation exchange resin, performing nanofiltration and concentration with a nanofiltration membrane, and adjusting a pH value with a sodium hydroxide solution to obtain a concentrated aqueous solution of a reduced β-nicotinamide mononucleotide disodium salt;
(4) adding a poor solvent to the concentrated aqueous solution of the reduced β-nicotinamide mononucleotide disodium salt;
(5) stirring and adding a seed crystal;
(6) continuously stirring to generate a suspended precipitate;
(7) continuously adding the poor solvent to increase a yield; and
(8) filtering and drying to obtain an amorphous compound of the reduced β-nicotinamide mononucleotide disodium salt,
wherein the poor solvent in the steps (4) and (7) is ethanol.

2. The preparation method according to claim 1, wherein in the step (1), an aqueous solution of the β-nicotinamide mononucleotide is first formulated, the thiourea dioxide is added to the solution to initiate the reaction after the pH value is adjusted to 9.5-10.0 by adding an alkali, and the pH value is maintained at 9.5-10.0 during the reaction.

3. The preparation method according to claim 1, wherein in the step (4), a temperature of the concentrated aqueous solution of the reduced β-nicotinamide mononucleotide disodium salt is 0 °C-25 °C, and the poor solvent is 95% ethanol.

4. The preparation method according to claim 1, wherein the seed crystal is ribose, glucose, or a reduced β-nicotinamide mononucleotide disodium salt prepared and obtained by using ribose or glucose as a precipitation primer.

5. The preparation method according to claim 1, wherein a mass percentage concentration of the reduced β-nicotinamide mononucleotide disodium salt in the concentrated aqueous solution of the reduced β-nicotinamide mononucleotide disodium salt in the step (4) is 5%-15%.

6. The preparation method according to claim 5, wherein a reaction temperature in the step (1) is 39 °C-44 °C; and a drying temperature in the step (8) is 20 °C-35 °C.

7. The preparation method according to claim 4, wherein the poor solvent is 95% ethanol, and a mass of the poor solvent added in the step (4) is 1.5-4.0 times that of the concentrated aqueous solution of the reduced β-nicotinamide mononucleotide disodium salt; and a mass of the poor solvent added dropwise in the step (7) is 3.5-6.0 times that of the concentrated aqueous solution of the reduced β-nicotinamide mononucleotide disodium salt.

8. An amorphous compound of a reduced β-nicotinamide mononucleotide disodium salt obtained by the preparation method according to claim 1, as detected by a differential scanning calorimeter, having two characteristic glass transition temperatures at 63.69 °C-74.67 °C and 85.52 °C-131.99 °C, with peak values of the two characteristic glass transition temperatures being 68.78 °C and 112.93 °C, respectively; having a characteristic melting point temperature at 217.21 °C-241.88 °C, with a peak value of the characteristic melting point temperature being 231.70 °C; and as scanned by a laser confocal Raman spectrometer, having characteristic peaks at Raman shifts of 814 cm⁻¹ and 845 cm⁻¹.
